SEQUENCE LISTING (4 sequences)

SEQ ID NO: 1 - protein, 85 aa, Canis lupus familiaris
MALSSQTQAACLLLLLLASVASVSVLPHQTGQLTDLRAQDTAGAEAGLQPTLQLRRLRRRDTHFPICIFCCGCCKTPKCGLCCIT

SEQ ID NO: 2 - protein, 84 aa, Homo sapiens
  misc (25)..(84): hepcidin antimicrobial peptide proprotein
MALSSQIWAACLLLLLLLASLTSGSVFPQQTGQLAELQPQDRAGARASWMPMFQRRRRRDTHFPICIFCCGCCHRSKCGMCCKT

SEQ ID NO: 3 - protein, 25 aa, Canis familiaris
DTHFPICIFCCGCCKTPKCGLCCIT

SEQ ID NO: 4 - protein, 25 aa, Homo sapiens
DTHFPICIFCCGCCHRSKCGMCCKT

(19) United States
(12) Patent Application Publication
(10) Pub. No.: US 2010/0068737 A1
(43) Pub. Date: Mar. 18, 2010

(54) Enzyme-linked Immunosorbent Assay (ELISA) for Canine Hepcidin

(75) Inventors: Michael M. Fry, Knoxville, TN (US); Stephen A. Kania, Powell, TN (US); Seung J. Baek, Knoxville, TN (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: UNIVERSITY OF TENNESSEE RESEARCH FOUNDATION, Knoxville, TN (US)

(21) Appl. No.: 12/560,672

(22) Filed: Sep. 16, 2009

Related U.S. Application Data
(60) Provisional application No. 61/097,319, filed on Sep. 16, 2008.

(51) Int. Cl. G01N 33/535

(52) U.S. Cl. 435/7.92

(57) ABSTRACT

The present invention relates to the use of hepcidin-specific antibodies for the detection of hepcidin in biological samples for the diagnosis of diseases and disorders related to iron metabolism. Kits useful in screening various subjects suspected of having diseases or disorders associated with iron metabolism are also provided.

### CROSS-REFERENCE TO RELATED APPLICATION

This application claims the benefit of U.S. Provisional Application Ser. No. 61/097,319, filed. Sep. 16, 2008, the disclosure of which is hereby incorporated by reference in its entirety, including all figures, tables and amino acid or nucleic acid sequences.

### SUMMARY OF THE INVENTION

The present invention relates to the use of hepcidin-specific antibodies for the detection of hepcidin in biological samples for the diagnosis of diseases and disorders related to iron metabolism. Kits useful in screening various subjects suspected of having diseases or disorders associated with iron metabolism are also provided.

### BRIEF DESCRIPTION OF THE DRAWINGS

FIG. 1 illustrates a hepcidin ELISA titration for pooled sera samples obtained from dogs before and 3 hours after iron injection.

FIG. 2 depicts the results of ELISA assays on hepcidin levels in animals after iron injection (3 hours, 6 hours, 24 hours and 5 days after injection).

### DETAILED DESCRIPTION OF THE INVENTION

One aspect of the disclosed technology relates to methods of detecting hepcidin levels in an individual. Typical serum concentration of hepcidin in normal people is between about 17-286 ng/mL. Levels that are above or below this range are associated with diseases or disorders that involve dysregulated iron metabolism.

Diseases or disorders associated with abnormal iron metabolism in humans include, and are not limited to: hemosiderosis, hemochromatosis, aceruloplasminemia, hypotransferrinemia, atransferrinemia, iron overload diseases, alcoholic liver diseases, nonalcoholic steatohepatitis, chronic hepatitis B and C infections, sideroblastic anemia, thalassemia, leukemia, macrocytic, microcytic or normocytic anemia, anemia with reticulocytosis, hemolytic anemia, bacterial or viral infection, inflammation, sepsis, anemia of chronic disease, neoplasia, and iron deficiency.

The term “hepcidin” relates to any mammalian hepcidin polypeptide known in the art. Non-limiting examples of such polypeptides include canine hepcidin (SEQ ID NO: 1) and human hepcidin (SEQ ID NO: 2). Other hepcidin polypeptides are known in the art and the amino acid sequences can be found in public databases such as GenBank and EMBL. Hepcidin polypeptides suitable for use as control samples for quantification of hepcidin in a biological sample can be obtained from commercial sources or recombinantly according to methods known in the art. For example, hepcidin encoding nucleic acids can be synthesized, cloned and expressed in appropriate host cells.

Various procedures are known in the art for the production of antibodies to polypeptides of interest. Thus, antibodies specific to the hepcidin polypeptide can be produced that specifically bind to amino acids 1-84 of either canine or human hepcidin. Some embodiments provide for the production of antibodies to one or more of the following subsequences of human hepcidin: a) amino acids 25-84 of SEQ ID NO: 2; b) amino acids 60-84 of SEQ ID NO: 2; c) amino acids 63-84 of SEQ ID NO: 2; d) amino acids 65-84 of SEQ ID NO: 2; and/or

| | | |
|---|---|---|
| i) | DTHFPICIFCCGCCKTPKCGLCCIT | (SEQ ID NO: 3) |

(a canine hepcidin peptide); or

| | | |
|---|---|---|
| ii) | DTHFPICIFCCGCCHRSKCGMCCKT | (SEQ ID NO: 4) |

(a human hepcidin peptide).

As discussed herein, antibodies include but are not limited to polyclonal, monoclonal, chimeric, single chain, Fab fragments and antibodies from Fab expression libraries. Various embodiments provide for the production of antibodies in various host animals; however, at least one of the antibodies used in the methods disclosed herein must be of avian origin (e.g., egg yolk antibodies or antibodies isolated from avian blood/serum). In certain aspects of the invention, the first and second antibodies used in the immunoassays disclosed herein bind to separate non-overlapping epitopes of the hepcidin polypeptide. Other aspects of the invention provide for the affinity purification of the hepcidin specific antibodies prior to their use in immunoassays.

Egg yolk antibodies can also be referred to as “IgY polyclonal antibody(ies)” or “IgY” or “IgY antibodies”. IgY antibodies can be purified from the egg yolk of an avian species. As used herein, the phrase “avian species” refers to any bird. One specific embodiment provides for the purification or isolation of IgY antibodies from chicken eggs.

IgY antibodies can be made in birds, preferably chickens in certain embodiments. Briefly, birds are injected with a purified protein (e.g., hepcidin) that acts as an antigen in the bird and resulting in the production of IgY antibodies that will bind with the protein. Gene-specific IgY antibodies can also be made by injecting gene expression vectors into birds for the in situ production of antigen within the bird. IgY can be collected from the yolks of bird eggs employing standard separation techniques. One additional aspect of the invention provides for the isolation of IgY antibodies specific for hepcidin by antigen affinity purification employing similar procedures to the antigen affinity purification of IgG. Affinity purified IgY antibodies can then be used in the immunoassays disclosed herein for the detection of hepcidin in biological samples.

Yet another aspect of the invention provides reagents (kits) for use in diagnostic assays for the detection of a hepcidin protein from individuals with hemochromotosis, iron deficiency anemia, hemosiderosis, liver cirrhosis other diseases associated with dysregulated iron metabolism. Kits provided herein typically contain a first and second antibody specific for hepcidin and at least one of these antibodies is of avian origin. Additionally, one or the antibodies provided in the kit can be labeled and the other antibody can be bound to a solid support in various embodiments provided herein. Optionally, the kits can also contain buffers and substrates that can be acted upon by an enzyme label to provide a detectable signal.

Any immunoassay system known in the art including, but not limited to: radioimmunoassays, enzyme-linked immunosorbent assay, “sandwich” assays, precipitin reactions, gel diffusion immunodiffusion assays, agglutination assays, fluorescent immunoassays, protein A immunoassays and immunoelectrophoresis assays can be used to detect the presence and/or amounts (levels) of hepcidin in a biological sample. Non-limiting examples of biological samples include blood, serum, plasma, urine, spinal fluids, other body fluids or tissue samples. These biological sample can, thus, provide for the identification of subjects (mammals, including humans) with hemochromotosis, iron deficiency anemia, hemosiderosis, liver cirrhosis and other diseases described herein. As discussed above, at least one of the antibodies used in the immunoassays provided herein is of avian origin. In one embodiment, a sample of blood can be removed from a subject and contacted with standard anti-coagulants. Plasma/serum can be isolated by methods known in the art and contacted with antibodies as described herein.

For example, a “sandwich” assay can be used to detect the amount of hepcidin in a biological sample. In this aspect of the invention, an unlabeled antibody is immobilized on a solid substrate (e.g., microtiter plate wells or another solid support) and the sample to be tested is brought into contact with the bound molecule for a period of time sufficient to allow formation of an antibody-antigen complex. A second antibody, labeled with a reporter molecule capable of inducing a detectable signal or unlabeled, is then added to the mixture contained on or in the solid substrate and contacted for a period of time sufficient to allow for the formation of a second antibody-antigen complex (to form an antibody-hepcidin-antibody complex). Unreacted material can then be washed away from the antibody-hepcidin-antibody complex and the presence of hepcidin is determined by observation of a signal, which may be quantitated by comparison with a control sample containing known amounts of hepcidin. For example, if a labeled antibody is used, the signal can be detected directly via the addition of a substance that allows for the generation of a signal. If an unlabeled antibody is used as the second antibody, than a third antibody (specific for the second antibody that is bound to hepcidin) that is labeled is used to contact the antibody-hepcidin-antibody complex to generate a signal. Simultaneous assays (where both sample and antibody are added simultaneously to the solid substrate-bound antibody) or reverse sandwich assays (where the labeled antibody and sample to be tested are first combined, incubated and added to the unlabelled surface bound antibody) can be used for the detection of hepcidin in biological samples.

As discussed above, a first antibody is bound to a solid support. The solid support can be glass or a polymer, including, but not limited to cellulose, polyacrylamide, nylon, polystyrene, polyvinylchloride or polypropylene. The solid supports may be in the form of tubes, beads, discs microplates, or any other surfaces suitable for conducting an immunoassay. After attachment of the first antibody to the solid support, the solid support is washed and a biological sample containing hepcidin is then added to the antibody-containing solid support for a period of time sufficient to allow binding of any hepcidin protein present to the antibody bound to the solid support. A second antibody can then be added to and incubated for an additional period of time sufficient to allow the second antibody to bind to the hepcidin polypeptide bound by the antibody attached to the solid support. The second antibody can be linked to a label that allows of the generation of a signal or the second antibody can be unlabeled. If the second antibody is unlabelled, a labeled third antibody that specifically binds to the second antibody can be used to detect the antibody-hepcidin-antibody complex formed in an immunoassay.

Non-limiting examples of labels suitable for use in this aspect of the invention include radioisotopes, enzymes or fluorophores. Commonly used enzymes include horseradish peroxidase, glucose oxidase, β-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product. In all cases where the second antibody is labeled, the labeled antibody is added to the first antibody-hepcidin protein complex and allowed to bind to the complex, and then the excess reagent is washed away. A solution containing the appropriate substrate is then added for the development of a signal.

Following are examples which illustrate procedures for practicing the invention. These examples should not be construed as limiting. All percentages are by weight and all solvent mixture proportions are by volume unless otherwise noted.

### Example 1

### Hepcidin ELISA Titration

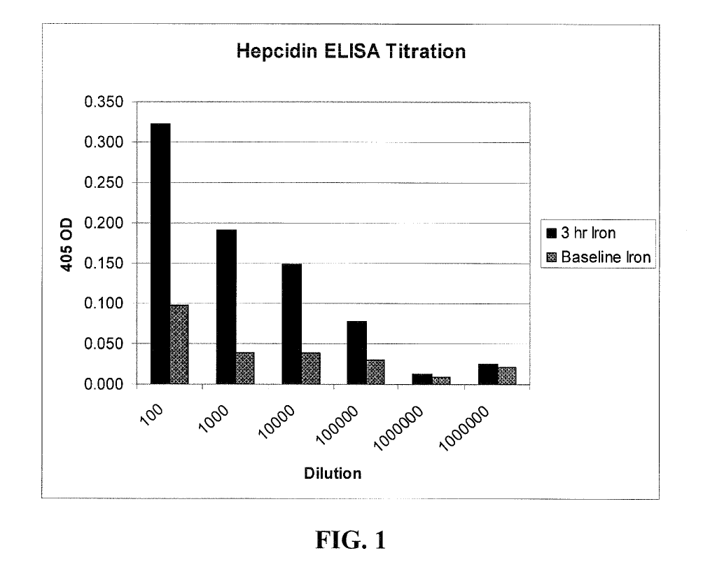

Immunol 2 96 well plates were coated with affinity purified rabbit anti-canine hepcidin diluted 1:250 in phosphate buffered saline (PBS). After the coating, and following each subsequent step, plates were washed three times with PBS containing 0.05% Tween 20 (PBSTW). Unbound sites on the plates were blocked with 1% fetal bovine serum prepared in PBSTW. Pooled sera samples obtained from dogs prior to iron injection and three hours after receiving iron were diluted in PBSTW containing 1% FBS at the reciprocal of the dilutions indicated (FIG. 1) and added to the wells. Affinity purified chicken anti-canine hepcidin diluted 1:250 in PBSTW was then added to the wells and incubated for one hour. This was followed by peroxidase conjugated affinity purified anti-chicken IgG diluted 1:500 in PBSTW (also incubated in the plate wells for one hour). TMB substrate was then added followed by 0.18 M H₂SO₄ to stop the reaction and color development. Plates were read at 450 nm in an EIA plate reader and values shown are averages of duplicate wells.

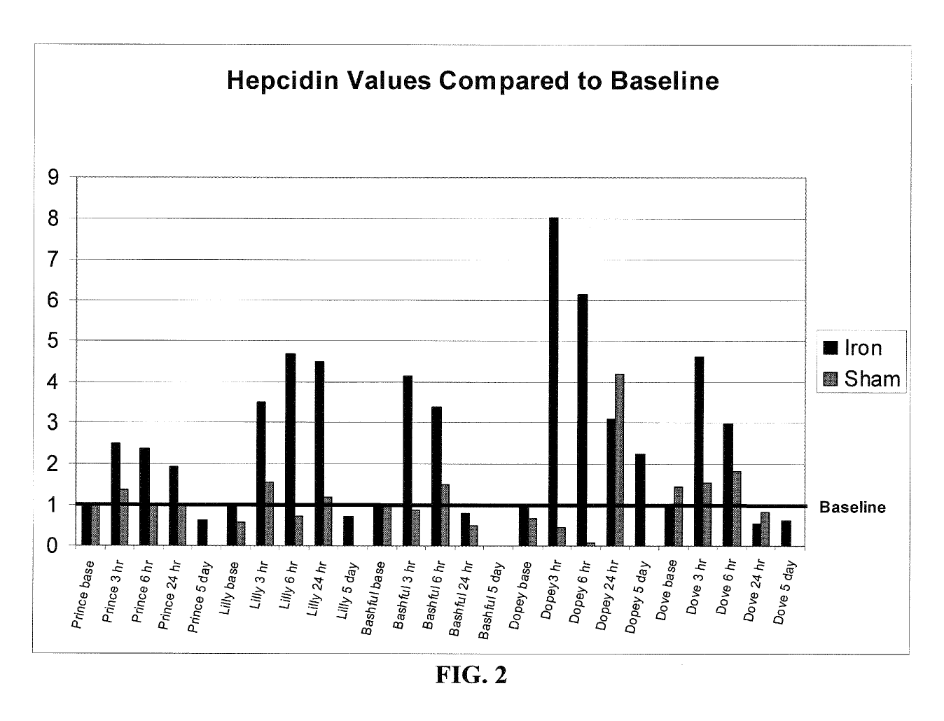

A second set of ELISA assays was conducted using a similar protocol to monitor hepcidin values in animals after administration of either iron or a sham composition. Immunol 2, 96 well plates were coated with affinity purified rabbit anti-canine hepcidin diluted 1:250 in phosphate buffered saline (PBS). After the coating, and following each subsequent step, plates were washed three times with PBS containing 0.05% Tween 20 (PBSTW). Unbound sites on the plates were blocked with 1% fetal bovine serum prepared in PBSTW. Serum was collected from animals prior to treatment and after receiving iron or sham treatment at the indicated times (FIG. 2). The collected serum was diluted 1:100 in PBSTW containing 1% FBS and incubated in the plate wells for one hour. After washing, affinity purified chicken anti-canine hepcidin diluted 1:250 in PBSTW was then added to the wells and incubated for one hour. This was followed by washing of the plates and the subsequent addition of peroxidase conjugated affinity purified anti-chicken IgG (diluted 1:500 in PBSTW) which was then incubated for one hour. After washing, TMB substrate was then added followed by 0.18 M H₂SO₄ to stop the reaction and color development. Plates were read at 450 nm in an EIA plate reader. Values shown are averages of triplicate wells (see FIG. 2).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. An immunoassay for detecting the presence of hepcidin in a biological sample comprising:
(a) contacting a biological sample with a first antibody and a second antibody to form an antibody-hepcidin-antibody complex; and
(b) detecting the amount of said hepcidin in said biological sample;
wherein:
at least one of said first or said second antibody is immobilized on a solid support;
said first and said second antibody bind non-overlapping epitopes;
at least one of said first or said second antibody is an avian antibody specific for hepcidin; and
the antibody that is not immobilized on said solid support is labeled or unlabeled.

2. The method according to claim 1, wherein:
a) said first antibody is an avian antibody specific for hepcidin and is immobilized on a solid support and said second antibody is a murine, goat or rabbit antibody specific for hepcidin that is unlabeled; or
b) said first antibody is a murine, goat or rabbit antibody specific for hepcidin and is immobilized on a solid support and said second antibody is an avian antibody specific for hepcidin that is unlabeled.

3. The method according to claim 1, wherein:
a) said first antibody is an avian antibody specific for hepcidin and is immobilized on a solid support and said second antibody is a murine, goat or rabbit antibody specific for hepcidin that is labeled; or
b) said first antibody is a murine, goat or rabbit antibody specific for hepcidin and is immobilized on a solid support and said second antibody is an avian antibody specific for hepcidin that is labeled.

4. The method according to claim 2, further comprising contacting said unlabeled antibody with a labeled antibody that specifically binds to said unlabeled antibody to detect the formation of said antibody-hepcidin-antibody complex.

5. The method according to claim 2, wherein said murine, goat or rabbit antibody is a monoclonal antibody specific for hepcidin.

6. The method according to claim 3, wherein said murine, goat or rabbit antibody is a monoclonal antibody specific for hepcidin.

7. The method according to claim 2, wherein said murine, goat or rabbit antibody is a polyclonal antibody specific for hepcidin.

8. The method according to claim 3, wherein said murine, goat or rabbit antibody is a polyclonal antibody specific for hepcidin

9. The method according to claim 2, wherein said avian antibody is a chicken antibody specific for hepcidin.

10. The method according to claim 2, wherein said avian antibody is an egg yolk antibody specific for hepcidin.

11. The method according to claim 1, wherein both of said antibodies are avian antibodies specific for hepcidin.

12. The method according to claim 1, wherein said antibodies are specific for canine hepcidin (SEQ ID NO: 1).

13. The method according to claim 1, wherein said antibodies are specific for human hepcidin (SEQ ID NO: 2).

14. The method according to claim 13, wherein said antibodies are specific for human hepcidin comprising: a) amino acids 25-84 of SEQ ID NO: 2; b) amino acids 60-84 of SEQ ID NO: 2; c) amino acids 63-84 of SEQ ID NO: 2; and/or d) amino acids 65-84 of SEQ ID NO: 2.

15. The method according to claim 1, wherein said first and said second antibodies are affinity purified prior to use in said immunoassay.

16. A kit comprising a first antibody and a second antibody that specifically bind to hepcidin, wherein at least one of said first or said second antibody is an avian antibody specific for hepcidin and wherein said first and/or said second antibody is, optionally, affinity purified.

17. The kit according to claim 16, wherein one of said first or said second antibody is immobilized on a solid support.

18. The kit according to claim 16, wherein one of said first or said second antibody is labeled.

19. The kit according to claim 16, wherein said antibodies are specific for canine hepcidin (SEQ ID NO: 1).

20. The kit according to claim 16, wherein said antibodies are specific for human hepcidin (SEQ ID NO: 2).

21. The kit according to claim 20, wherein said antibodies are specific for human hepcidin comprising: a) amino acids 25-84 of SEQ ID NO: 2; b) amino acids 60-84 of SEQ ID NO: 2; c) amino acids 63-84 of SEQ ID NO: 2; and/or d) amino acids 65-84 of SEQ ID NO: 2.
